(19) United States
(12) Patent Application Publication
(10) Pub. No.: US 2025/0002833 A1
(43) Pub. Date: Jan. 2, 2025

(54) ENVIRONMENT CONTROLLED CHAMBER WITH IR CONDENSATION REDUCTION

(71) Applicant: Percival Scientific, Inc, Perry, IA (US)

(72) Inventors: Henry Imberti, Boone, IA (US); David Imberti, Boone, IA (US); Daniel Kiekhaefer, Johnston, IA (US)

(73) Assignee: Percival Scientific, Inc, Perry, IA (US)

(21) Appl. No.: 18/669,905

(22) Filed: May 21, 2024

Related U.S. Application Data
(60) Provisional application No. 63/511,090, filed on Jun. 29, 2023.

(51) Int. Cl. C12M 1/00; C12M 3/00

(52) U.S. Cl. CPC C12M 41/14; C12M 31/10; C12M 41/06; C12M 3/00

(57) ABSTRACT

An environment controlled chamber is provided. The environment controlled chamber includes an insulated cabinet, a refrigeration system adapted for cooling the insulated cabinet, a control system, a plurality of lighting elements for providing light within a compartment of the insulated cabinet, and a plurality of infrared elements for providing heating within the insulated cabinet. The control system is configured to control the refrigeration system and the lighting elements to provide environmental conditions according to user settings for promoting photosynthetic tissue culture growth. The control system is configured to control the plurality of infrared lighting elements to selectively apply heating to assist with preventing condensation on petri dishes stored within the insulated cabinet.

### RELATED APPLICATIONS

The present application claims priority to U.S. Provisional Patent Application No. 63/511,090, filed Jun. 29, 2023, hereby incorporated by reference in its entirety.

### FIELD OF THE INVENTION

The present invention relates to environmental controlled chambers such as plant growth chambers, tissue culture chambers, incubators, and the like. More particularly, but not exclusively the present invention relates to environment controlled chambers configured to reduce condensation. The present applications

### BACKGROUND OF THE ART

Environment controlled chambers are widely used including in the context of plant studies related to plant growth, incubation, germination and other purposes. The term “environment controlled chamber” is intended to include plant growth chambers, tissue culture chambers, germinators, incubators, and other variations of environment controlled chambers. In plant growth research, parameters such as temperature, light, and humidity (as well as other parameters such as CO₂) are tightly controlled.

Light is one of the most important parameters studied in the field of plant growth research as it is a primary component in the process of photosynthesis. In some species of plants it is critical to have stable light irradiance throughout the entire chamber temperature operating range (typically about 4° C. to 45° C.). Not all light is considered to contribute to plant growth or photosynthesis, what is of interest is the photosynthetically active radiation (PAR) spectrum which ranges from 400 to 700 nanometers and corresponds to the visible light spectrum, as well as some other light with a strong influence on the plant including ultraviolet (UV) between 380-400 nm and far red between 700-780 nm. For plant tissue culture, light levels are generally much lower than those for plant growth.

In the context of plant studies, condensation on petri dishes is a common issue that can affect the results of experiments, introduces an unknown variable to the experiment, and the ability to observe the progress of experiments without removing the cover of a petri dish. Unfortunately, avoiding condensation is inconsistent with the requirements of environment controlled chambers. In a controlled environment of the environment controlled chamber, different temperature and lighting conditions may be cycled through so as to simulate day and night cycles of natural growing conditions. This change in conditions results in condensation on the bottom surface of the top cover of the petri dishes. This is inconvenient for plant researchers and it would be advantageous to reduce or eliminate such condensation.

### SUMMARY

Therefore, it is a primary object, feature, or advantage to improve over the state of the art.

It is a further object, feature, or advantage to provide environment controlled chambers suitable for use in plant studies.

It is a still further object, feature, or advantage to provide environment controlled chambers which reduce or eliminate condensation such as condensation at bottom surfaces of top covers of petri dishes.

One or more of these and/or other objects, features, or advantages will become apparent from the specification and claims that follow.

According to one aspect, an environment controlled chamber is provided. The environment controlled chamber includes an insulated cabinet, a refrigeration system adapted for cooling the insulated cabinet, a control system, a plurality of v lighting elements for providing light within a compartment of the insulated cabinet, and a plurality of infrared lighting elements for providing heating within the insulated cabinet. The control system is configured to control the refrigeration system and the lighting elements to provide environmental conditions according to user settings for promoting photosynthetic tissue culture growth. The control system is configured to control the plurality of infrared lighting elements to selectively apply heating to assist with preventing condensation on petri dishes stored within the insulated cabinet. The control system may be configured to control the lighting elements to provide the environmental conditions simulating a day to night transition and the control system may activate the infrared lighting elements based on timing of the day to night transition. The control system may be configured to activate the infrared lighting elements in order to add heat within the insulated cabinet while cooling the insulated cabinet to thereby limit rate of temperature change within the insulated cabinet to assist with preventing the condensation on the petri dishes stored within the insulated cabinet. The control system may also be configured to limit temperature offsets using the infrared light. The environment controlled chamber may further include a user interface configured to receive input from a user to establish the user settings. The environment controlled chamber may further include a plurality of sensors operatively connected to the control system. The environmental controlled growth chamber may further include a LED array wherein the LED array includes a set of LEDs for providing lighting and a set of LEDs for providing infrared lighting. The environment controlled chamber may include a shelf and the LED array may be mounted above the shelf. The control system may be configured to control the plurality of infrared lighting elements to increase the difference between the surface temperature of the petri dish and the dew point inside the petri dish. In some embodiments, a material may be integrated into a medium of the petri dishes to increase heating effects of the plurality of infrared lighting elements. In some embodiments, a material may be integrated into the petri dishes or its surroundings to increase heating effects of the plurality of infrared lighting elements. In some embodiments, a material is integrated into the compartment to increase heating effects of the plurality of infrared lighting elements.

According to another aspect, a method for controlling an environment controlled chamber to assist with preventing condensation on an inside of a top cover of a petri dish stored within the environment controlled chamber is provided. The method includes steps of receiving through a user interface parameters for controlling the environment controlled chamber where the parameters including a temperature setting, and a light control setting associated with activating and deactivating lighting elements within the environment controlled chamber. The method further includes activating infrared lighting elements within the environment controlled chamber based on the temperature setting and the light control setting and while cooling the environment controlled chamber to increase temperature differential between a surface temperature of the top cover of the petri dish and an ambient dew point inside the petri dish. The infrared lighting elements may be infrared LEDs. The method may further include a step of acquiring sensor measurements from a plurality of sensors and wherein the activating the infrared lighting elements is further based on the sensor measurements.

According to another aspect, an environment controlled chamber system includes an insulated cabinet, a refrigeration system adapted for cooling the insulated cabinet, a control system, a plurality of light emitting diodes (LEDs) for providing light within a compartment of the insulated cabinet, and a plurality of infrared (IR) light emitting diodes (LEDs) for providing heating within the insulated cabinet. The control system is configured to control the refrigeration system and the LEDs to provide environmental conditions according to user settings for promoting photosynthetic tissue culture growth. The control system may be configured to control the plurality of IR LEDs to selectively apply heating to assist with preventing condensation on petri dishes stored within the insulated cabinet by increasing differences between surface temperature of a petri dish and a dew point inside the petri dish. The control system may be configured to control the LEDs to provide the environmental conditions simulating a day to night transition and wherein the control system activates the IR LEDs based on timing of the day to night transition. The environment controlled chamber system may be configured to activate the IR LEDs in order to add heat within the insulated cabinet while cooling the insulated cabinet to thereby limit rate of temperature change within the insulated cabinet to assist with preventing the condensation on the petri dishes stored within the insulated cabinet. The environment controlled chamber system may further include a user interface configured to receive input from a user to establish the user settings. In some embodiments, a material may be integrated into a medium of the petri dishes to increase heating effects of the plurality of IR LEDs. In some embodiments, a material may be integrated into the petri dishes or its surroundings to increase heating effects of the plurality of IR LEDs. In some embodiments, a material may be integrated into the compartment to increase heating effects of the plurality of IR LEDs.

According to another aspect, an environment controlled chamber includes an insulated cabinet, a refrigeration system adapted for cooling the insulated cabinet, a control system, a plurality of lighting elements within a compartment of the insulated cabinet, and a plurality of infrared elements for providing heating within the insulated cabinet. The control system is configured to control the refrigeration system and the lighting elements to provide environmental conditions according to user settings. The control system is configured to control the plurality of infrared elements to selectively apply heating to assist with preventing condensation on petri dishes stored within the insulated cabinet.

### BRIEF DESCRIPTION OF THE DRAWINGS

FIG. 1 is an image acquired using an infrared camera to show surface temperature of a petri dish without the use of IR LEDs.

FIG. 2 is an image acquired using an infrared camera to show the surface temperature of the petri dish when IR LEDs are used.

FIG. 3 is an image of a petri dish where IR LEDs are not used in order to show condensation on the bottom surface of the top cover of a petri dish.

FIG. 4 is an image of the petri dish where IR LEDs are used in order to show the condensation pattern on the bottom surface of the top cover of the petri dish.

FIG. 5 illustrates one example of an environment controlled chamber.

FIG. 6 illustrates an interior compartment of the environment controlled chamber with shelving and petri dishes supported on the shelving.

FIG. 7 illustrates one example of a panel which includes both lighting elements and infrared lighting elements.

FIG. 8 is a block diagram illustrating one example of the environment controlled chamber.

FIG. 9 illustrates one example of a method.

### DETAILED DESCRIPTION

In order to reduce or eliminate petri dish condensation in environment controlled chambers, infrared (IR) lighting is used to increase the temperature difference between surface and air temperatures. By increasing the surface temperature in this manner, psychrometrically, this slight increase in temperature allows heating to just above the dew point, or possibly avoid temperature offset issues with infrared caused by moving between day and night modes. At the same time, heating was sufficiently minimal so as not to adversely affect growing conditions.

In order to confirm the effect of infrared radiation on petri dish condensation within environment controlled chambers, a study was performed. In this study, a model ARC-36L5 chamber, specifically SO #32131 (available from Percival Scientific, Perry, Iowa), was selected. This chamber is a CU chamber that presents ideal testing conditions for our purposes, given that these models of chambers are where we have experienced previous condensation issues. The five tiers of shelving in the chamber were outfitted with a combination of standard 2′×1′ SciWhite (SW) tiles and SW+IR tiles. The first, second, fourth, and fifth tiers each contained two standard SW tiles. The third tier contained a SW+IR tile. These selections were designed to match fluorescents lighting output better as fluorescent lighting output does not appear to have the same level of condensation issues. The IR lighting system utilized in this experiment had an equal number of infrared and white LEDs and ran at an approximate output of 13 W of infrared. Of course, different numbers or relative numbers of infrared LEDs and white LEDs may be used and different levels of output for the infrared LEDs may also be used.

The initial temperature was set to 28° C. and held there, after which two different types of experiments were conducted. In both experiments, all lights were turned off except infrared and the temperature was decreased to 20° C. However, the temperature control method varied between the two experiments. In the first experiment (Experiment One), the temperature was ramped down slowly over two hours, while in the second experiment (Experiment Two), there was no ramping. Pictures of the condensation were collected in both experiments, but planimeter data was only captured in the second experiment. The specific tiles and IR lighting system were consistent across both experiments.

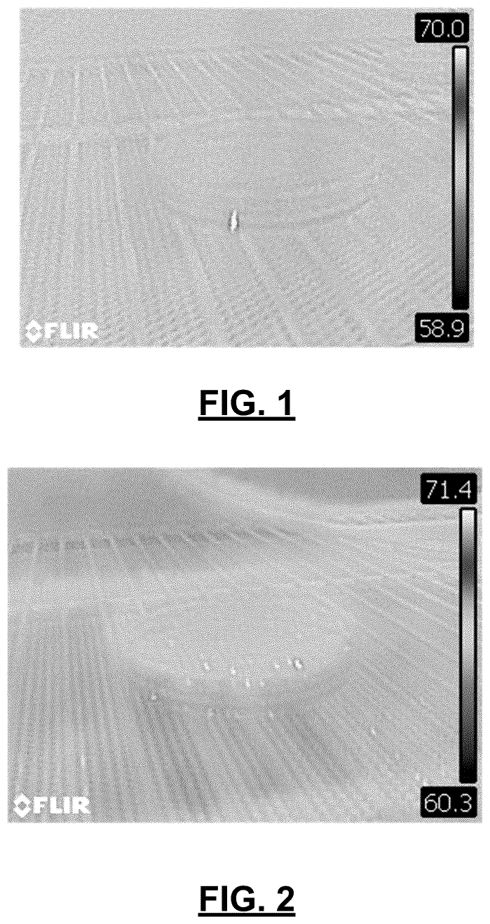

To analyze the difference between surface temperature and air temperature effects, we first verified conditions with an infrared camera. FIG. 1 is an image acquired using an infrared camera to show surface temperature of a petri dish without the use of IR LEDs. FIG. 2 is an image acquired using an infrared camera to show the surface temperature of the petri dish when IR LEDs are used.

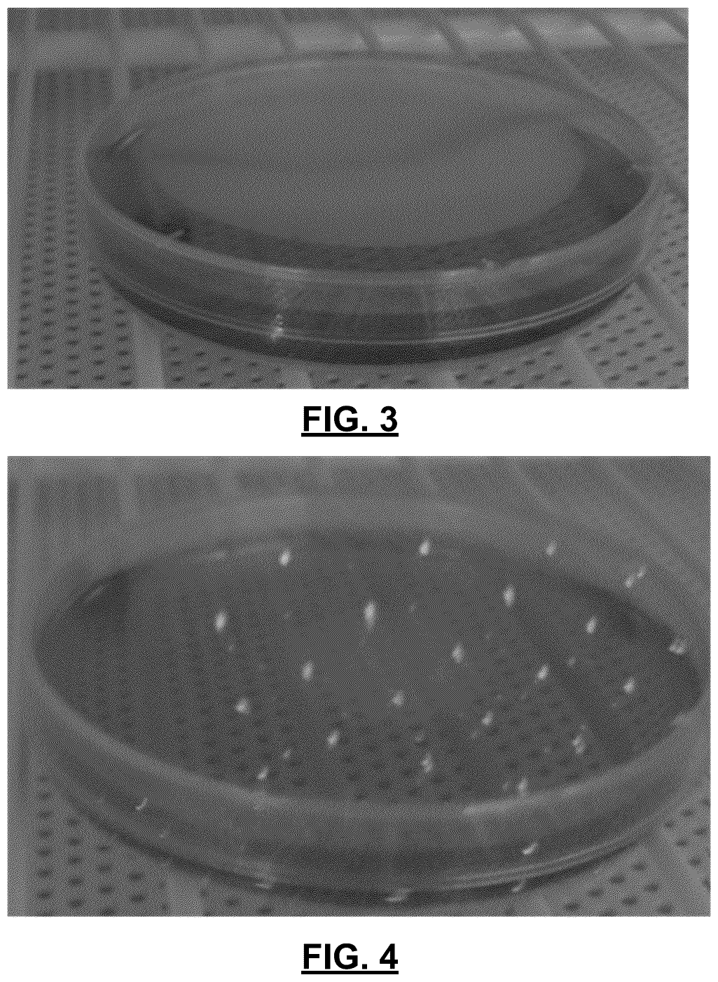

After holding at 20 C for a day in Experiment One, FIG. 3 is an image of a petri dish where IR LEDs are not used in order to show condensation on the bottom surface of the top cover of a petri dish. FIG. 4 is an image of the petri dish where IR LEDs are used in order to show the condensation pattern on the bottom surface of the top cover of the petri dish. Note that the camera used did detect some of the IR LEDs and not all cameras would do so.

Experiment One had two issues. One was that we were conflating both the ramping with the effect of IR LEDs. The other is that there is still some condensation on the IR petri dishes, so we wanted a way to demonstrate effects beyond qualitative observation. Because of this, we used a planimeter to measure the area of the condensation patterns on the petri dishes throughout the chamber in Experiment Two. The following table summarizes these results.

| | Rear- | Front- | | Rear- | Front- | | |
|---|---|---|---|---|---|---|---|
| | Left | Left | Center | Right | Right | | |
| Tier | (in\hat{ }2) | (in\hat{ }2) | (in\hat{ }2) | (in\hat{ }2) | (in\hat{ }2) | Av | Comments |
| 1 | 5.838 | 0.432 | 3.546 | 4.454 | 4.043 | 3.66 | Front-Left |
| | | | | | | | evaporated |
| | | | | | | | quickly, |
| | | | | | | | best |
| | | | | | | | estimate. |
| | | | | | | | Started at |
| | | | | | | | 2:20 |
| 2 | 4.238 | 2.703 | 2.681 | 4.692 | 2.141 | 3.29 | |
| 3 | 0.432 | 0 | 0 | 1.038 | 2.076 | 0.71 | Infrared |
| | | | | | | | Tier. |
| | | | | | | | Started at |
| | | | | | | | 2:31 |
| 4 | 4.778 | 4.562 | 4 | 3.892 | 4.216 | 4.29 | |
| 5 | 0 | 4.432 | 0.778 | 2.551 | 3.611 | 2.84 | Finished |
| | | | | | | | at 2:43 |
| Av | 3.82 | 2.43 | 2.2 | 3.33 | 3.22 | 2.96 | |

Our results revealed that the IR camera indicated higher surface temperatures on the petri dishes, verifying the surface temperature/air temperature distinction. Additionally, a difference of 1° C. was observed with the IR camera. This provides us with an extra 5% RH clearance in the dish interior, which helps clean off the petri dish much faster.

Our results for Experiment One showed that it appeared to perform better than the control. Further, in Experiment Two, the planimeter data provided a solid 3-fold improvement using IR chips. This finding was qualitatively backed up by the images acquired during the experiments.

Thus, the experiments have verified the surface temperature relative to the air temperature difference as the apparent cause of condensation in normal chamber operation, and that IR chips are effective in solving this issue. The planimeter data indicates a significant reduction of condensation, and the pictures also qualitatively back up our results.

In order to benefit from the use of infrared lighting, various methods may be performed, and systems may be used. This may include variations in the amount of infrared lighting, the timing of when the infrared lighting is activated, and the duration of the infrared lighting.

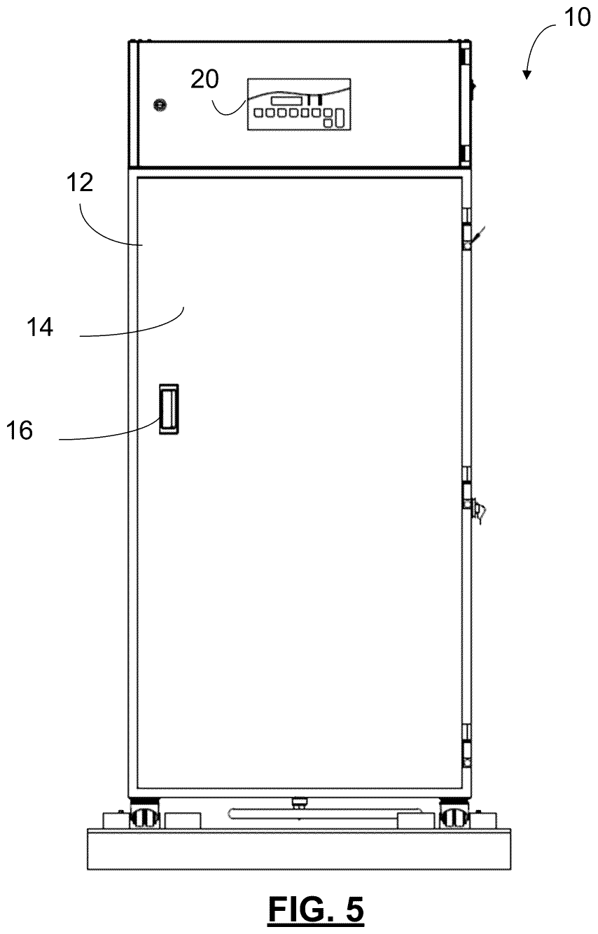

FIG. 5 illustrates one example of an environment controlled chamber 10. Additional examples of environment controlled chambers are shown in U.S. Pat. No. 7,794,105, hereby incorporated by reference in its entirety. The environment controlled chamber 10 has an insulated cabinet 12. A door 14 with handle 16 provides access to within the insulated cabinet 12. In some embodiments the environment controlled chamber 10 may have multiple doors. In some embodiments, the environment controlled chamber may be a walk-in chamber, or may be otherwise configured.

A user interface 20 is shown positioned above the door 14 of the insulated cabinet 12. The user interface 20 may be used to set conditions associated with the environment controlled chamber.

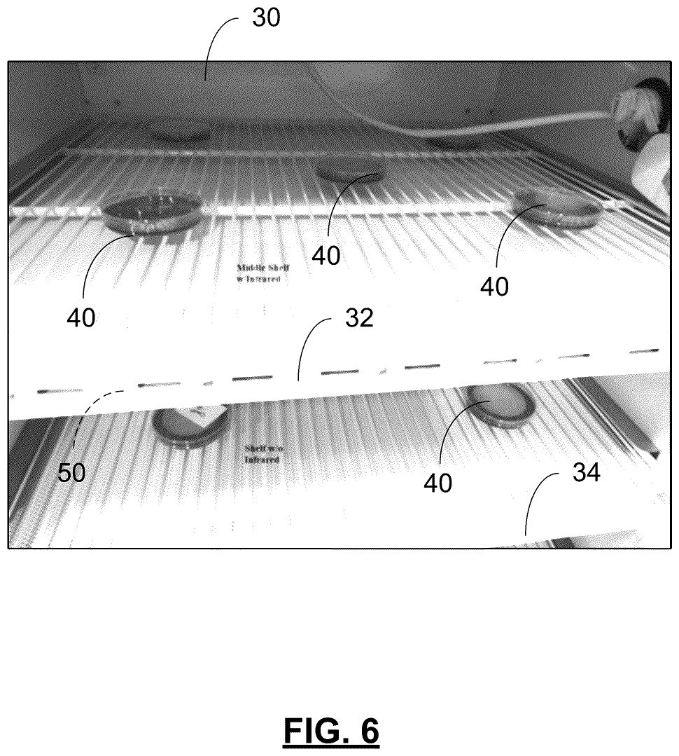

FIG. 6 illustrates an interior view of the environment controlled chamber 10. An interior compartment 30 may include one or more shelves 32, 34. Different experiments may be placed on the shelves 32, 34 including the petri dishes 40, shown.

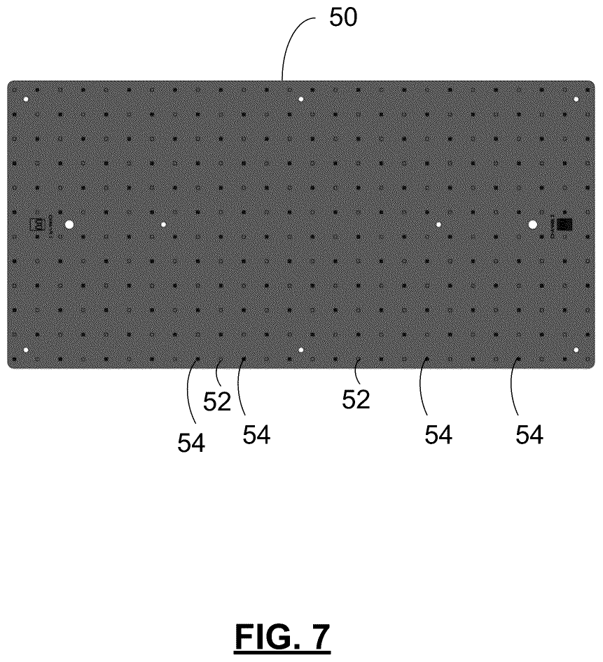

FIG. 7 illustrates one example of a panel 50 which may be mounted within the interior compartment such as at a top portion of the interior compartment or under a shelf within the interior compartment. Mounted on the panel 50 are a plurality of lighting elements in the form of lighting elements 52. The lighting elements may include LEDs to provide visible spectrum lighting and may further provide additional wavelengths of light including ultraviolet (UV), and far infrared lighting. There are also a plurality of infrared elements 54 which may be in the form of infrared LEDs. In some embodiments, the infrared elements 54 may be infrared heating elements.

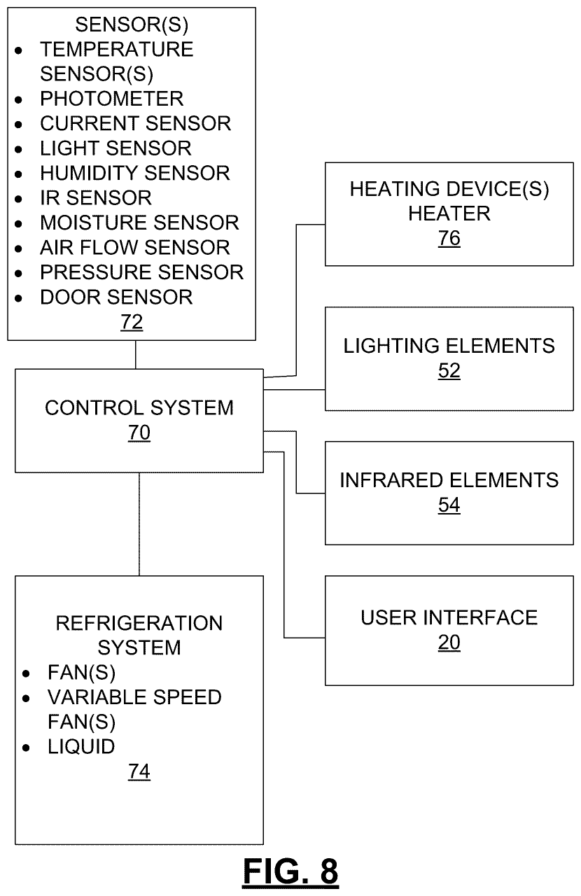

FIG. 8 is a block diagram of an environment controlled chamber 10. The environment controlled chamber 10 includes a control system 70. A plurality of sensors 72 may be operatively connected to the control system 70. Examples of sensors 72 may include without limitation, temperature sensors, photometers, current sensors, light sensors, humidity sensors, IR sensors, moisture sensors, air flow sensors, pressure sensors, and door sensors.

A humidity sensor can help monitor the moisture level inside the environment controlled chamber. High humidity levels can lead to condensation. An example of such a sensor is the capacitive humidity sensor, which measures the change in capacitance due to moisture absorption. Of course, other types of humidity sensors may be used.

As condensation occurs when warm air comes into contact with a cold surface, monitoring the temperature inside the environment controlled chamber can help identify temperature fluctuations that may cause condensation. Various types of temperature sensors may be used. Examples include thermocouples, RTDs (Resistance Temperature Detectors), and thermistors.

An IR sensor may be used to detect the presence of water or moisture by measuring the infrared radiation emitted by the water molecules. This can help to directly monitor condensation inside the environment controlled chamber.

A moisture sensor can detect the presence of water or moisture on surfaces. This can help to directly monitor condensation inside the environment controlled chamber. Examples of moisture sensors include resistive moisture sensors and capacitive moisture sensors.

Airflow sensors can be used to monitor air flow within the environment controlled chamber. Poor air circulation can cause condensation inside the environment controlled chamber. Monitoring the airflow inside the environment controlled chamber with an airflow sensor (such as a hot-wire anemometer) can help identify areas with poor circulation that may be prone to condensation.

Changes in pressure inside the environment controlled chamber can also lead to condensation. Thus, a pressure sensor can help monitor these changes and detect conditions that may cause condensation.

In addition, frequent opening and closing of the environment controlled chamber door can introduce warm, humid air into the environment controlled chamber, leading to condensation. A door sensor can help monitor the door's opening and closing frequency, and provide insights into whether this may be contributing to condensation issues.

Thus, one or more of these sensors or combinations of these sensors may be used to assist in providing data or measurements which may be used to assist in selectively activating infrared heating in order to reduce or avoid condensation of petri dish within the environment controlled chamber. It is to be understood that not all of these sensors need be present, but only a subset may be present. It is to be further understood that there may be multiple sensors of the same type positioned at different locations throughout the environment controlled chamber. For example, different shelves within the environment controlled chamber may have their own set of sensors. Alternatively, sensors may be positioned so as to more accurately determine useful information. For example, temperature sensors may be positioned closer to a surface where temperature is to be measured. In some embodiments, sensor measurements may be calibrated or offset to account for measuring at the location of a particular sensor as opposed to at a particular location of interest.

It should be further understood, that in some embodiments, for testing purposes a large number of sensors and/or a large number of different types of sensors may be present for observing results of experiments, but after collecting sufficient data to understand various relationships present, including, without limitation, relationships between condensation, temperature, humidity, dew point, temperature changes, day/night cycles, and other parameters have been identified or modeled, then control system parameters may be determined which do not require any additional sensors beyond what is conventionally available in an environment controlled chamber.

A refrigeration system 74 or cooling system may be operatively connected to the control system 70. The refrigeration system 74 may include, without limitation, fans, variable speed fans, and/or liquid cooling systems including those which use a compressor as a part of its cooling mechanism.

One or more heating devices 76 may be electrically connected to the control system 70 such as electric heaters or other types of heating devices. Such a heating device 76 is separate from any heating provided by the infrared lighting elements.

One or more lighting elements 52 such as LEDs may be operatively connected to the control system 70. The lighting elements 52 may provide lighting to simulate natural lighting across a range of frequencies conducive to plant growth. This may include lighting within the visible spectrum and may further include UV as well as far red light. The lighting elements 52 may be a part of one or more arrays. For example, there may be at least one separate array of LEDs for each shelf or tier within an interior compartment of the environment controlled chamber. It is contemplated that the lighting elements may provide for varying light intensity by color, such as up to 1850 μmoles/m2/s at 6 inches from the LEDs. For tissue cultures the light level may be rather low such as low as 50 μmoles/m2/s. It is further submitted that different tiers or areas within the chamber may be independently dimmable to provide enhanced control of light intensity. In addition, a wide range of programmable wavelengths may be used for specific stages of plant development.

One or more infrared elements 54 may be operatively connected to the control system 70. The infrared elements 54 which may be infrared LEDs may provide lighting not for any effect on tissue growth, but to assist in heating in a manner to avoid condensation of the top covers of Petri dishes. Note that only a relatively small heating effect is needed from the infrared LEDs. In addition, only a localized heating effect is needed relative to the placement of Petri dishes. Thus, in one embodiment, the LEDs may be placed directly over shelves so that infrared LED output is focused on where Petri dishes are located.

In some embodiments, the infrared level may be held constant within the chamber to avoid condensation. In other embodiments, the infrared lighting may be tuned and dimmed to the maximum intensity of light being run in the chamber. There is a linear relationship between the requisite amount of infrared light to avoid condensation and the maximum intensity of lighting being run in the chamber. Thus, the control system 70 may exercise control of the infrared lighting based on the maximum intensity of lighting being run in the chamber in order to avoid condensation.

A user interface 20 is also shown. The user interface 20 may be used so that a user may specify operating parameters for the environment controlled chamber 10. The user interface 20 may show current status of the chamber such as temperature, relative humidity, lighting, time, and other information regarding environmental conditions.

The user interface 20 may be used to program the environmental chamber by specifying a plurality of steps to perform. Each step may have an associated time step, which may indicate the time at which a step is to begin. The steps may include a temperature set point, a humidity setpoint, and light settings of light outputs. The light settings may indicate which lights are on or off or a desired level of light output for each light setting. The steps may indicate CO₂ levels. It is contemplated that the steps may also allow for direct control of the infrared LEDs such that a user may indicate a step which involves turning the infrared LEDs on or off or indicating a desired level of light output for each infrared LEDs. However, as will be further explained, it may be preferred to control the infrared LEDs without relying on user settings, but automatically in response to other conditions within the environmental chamber.

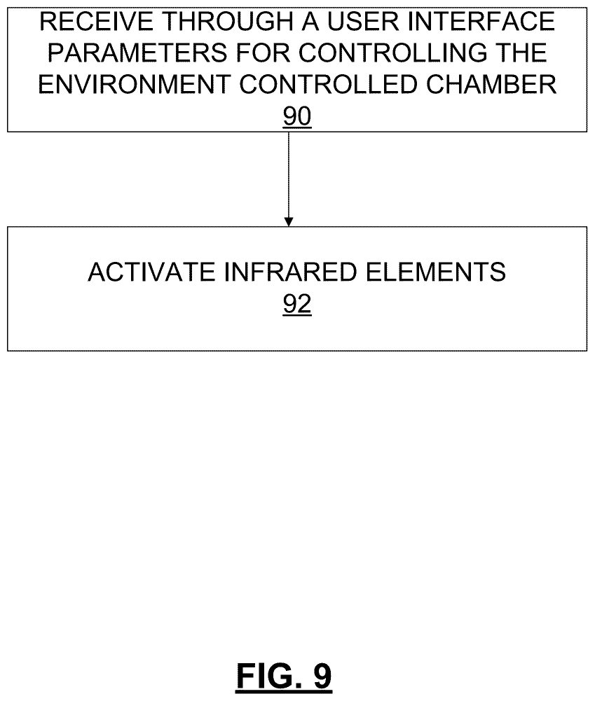

FIG. 9 illustrates one example of a method. In step 90, the environment controlled chamber receives through the user interface parameters for controlling the environment controlled chamber. These parameters may include the information provided by a user to program different steps by specifying the time, temperature set point, humidity setpoint, and light settings, or other parameters which may be used to control the environment of the environment controlled chamber.

Next in step 92, the infrared lighting elements are activated during control of the environmental chamber in order to prevent condensation of the inner surface of the top cover of at least one petri dish. The infrared lighting elements may be activated according to any number of different control methodologies.

In one example, user programming may specifically provide for activating the infrared lighting elements at different times. In another example, the infrared lighting elements may be activated based on one or more sensor readings reaching a threshold. For example, based on a temperature reaching a certain threshold, based on relative humidity reaching a certain threshold, based on a differential between two or more temperature sensor readings reaching a certain threshold, based on a rate of change of a sensor reading reaching a certain threshold, or otherwise. In another example, the infrared lighting elements may be activated based on a delay after a certain action is initiated or a certain condition occurs.

In some embodiments, a surface temperature may be measured directly and compared to air temperature which may also be measured directly. It is to be understood that various methods may be applied based on available sensor reading, relationships developed from measurements, in order to determining the timing and duration of the application of infrared heating through the infrared lighting elements such as infrared LEDs.

In addition, to these different variation, other variations are considered. For example, in one embodiment it is contemplated that in order to enhance the effects of infrared lighting, the agar in the petri dishes may be modified in order to enhance the heating effect of infrared lighting. Agar may be combined with nutrients, such as sugars, proteins, and minerals, in the conventional manner to provide a suitable growth medium for the specific microorganisms being studied. Additional additives may be added to the agar in order to enhance the effects of infrared lighting. In some embodiments, the additives do not alter or do not significantly alter effects of other frequencies of light, including visible light on tissue growth. The additives may be in the form of selective emitters. Such materials may have high absorption or reflection in the infrared region of the electromagnetic spectrum, while maintaining high transmission in the visible light region or in other portions of the light spectrum. In some embodiments, the materials may include infrared-reflective coatings. In other embodiments, the materials may include nanostructured materials such as photonic crystals or metamaterials.

Where such a material is used, it is contemplated that a user setting may be present which allows a user to indicate that the agar or other medium is enhanced with the material and the control system may take into account the effects of the material.

In other embodiments, it is contemplated that the Petri dish may be made of materials that enhance the effects of infrared lighting without adversely altering effects of visible light or other frequencies of light of interest on tissue growth. The additives may be in the form of selective emitters. Such materials may have high absorption or reflection in the infrared region of the electromagnetic spectrum, while maintaining high transmissivity in the visible light region or other regions of interest. In some embodiments, the materials may include infrared-reflective coating coatings. In other embodiments, the materials may include nanostructured materials such as photonic crystals or metamaterials. Some polymers, like polyethylene and polypropylene, are transparent to visible light but have high IR absorption or reflection properties. These are further examples of materials which may be used. Such materials may be used which still result in the Petri dish being optically transparent. Such materials may be added into glass, clear plastic, or other appropriate Petri dish material.

Where such a material is used, it is contemplated that a user setting may be present which allows a user to indicate that the petri dish is enhanced with the material and the control system may take into account the effects of the material.

Materials which affect the infrared heating may otherwise be included within the environment controlled chamber. The materials may be integrated into shelving, racks, or other interior surfaces within the environment controlled chamber in order to enhance the effects of infrared heating. Although this may be accomplished with various types of materials including those examples previously provided, one example is infrared-reflective coatings.

As shown, infrared lighting is used to increase the temperature difference between surface and air temperatures. The control system may be configured to increase the surface temperature just along the top of the petri dish. Psychrometrically, this slight increase in temperature can rise just above the dew point in order to prevent condensation. In addition, or alternatively temperature offset issues associated by moving between day and night modes may be avoided or attenuated with infrared. It is to be further understood that the environment controlled chambers shown and described may be used for various type of applications. For example, where the environment controlled chamber is a tissue culture chamber is it may be used for any number of types of biological tissue cultures including human tissue culture, insect tissue culture, or other types of tissue culture. In some embodiments, the lighting is not required.

Therefore, methods and systems have been shown and described which apply infrared light from infrared lighting elements such as infrared LEDs in environment controlled chambers to prevent or reduce condensation. Although various examples or embodiments have been shown and described throughout, the present disclosure is not to be limited to these specific embodiments.

What is claimed:

1. An environment controlled chamber, comprising:
an insulated cabinet;
a refrigeration system adapted for cooling the insulated cabinet;
a control system;
a plurality of lighting elements within a compartment of the insulated cabinet; and
a plurality of infrared lighting elements for providing heating within the insulated cabinet;
wherein the control system is configured to control the refrigeration system and the lighting elements to provide environmental conditions according to user settings for promoting photosynthetic tissue culture growth;
wherein the control system is configured to control the plurality of infrared lighting elements to selectively apply heating to assist with preventing condensation on petri dishes stored within the insulated cabinet.

2. The environment controlled chamber of claim 1 wherein the plurality of lighting elements provide at least one of visible light, ultraviolet light, and far red lighting.

3. The environment controlled chamber of claim 1 wherein the control system is configured to control the lighting elements to provide the environmental conditions simulating a day to night transition and wherein the control system activates the infrared lighting elements based on timing of the day to night transition.

4. The environment controlled chamber of claim 1 wherein the control system is configured to activate the infrared lighting elements in order to add heat within the insulated cabinet while cooling the insulated cabinet to thereby limit rate of temperature change within the insulated cabinet to assist with preventing the condensation on the petri dishes stored within the insulated cabinet.

5. The environment controlled chamber of claim 1 further comprising a user interface configured to receive input from a user to establish the user settings.

6. The environment controlled chamber of claim 1 further comprising a plurality of sensors operatively connected to the control system.

7. The environment controlled chamber of claim 1 further comprising a LED array wherein the LED array includes the lighting elements and the infrared lighting elements.

8. The environment controlled chamber of claim 7 further comprising a shelf and wherein the LED array is mounted above the shelf.

9. The environment controlled chamber of claim 1 wherein the control system is configured to control the plurality of infrared lighting elements to increase differences between surface temperatures of the petri dishes and dew points inside the petri dishes.

10. The environment controlled chamber of claim 1 wherein a material is integrated into a medium of the petri dishes to increase heating effects of the plurality of infrared lighting elements.

11. The environment controlled chamber of claim 1 wherein a material is integrated into the petri dishes and/or surroundings thereof to increase heating effects of the plurality of infrared lighting elements.

12. The environment controlled chamber of claim 1 wherein a material is integrated into the compartment to increase heating effects of the plurality of infrared lighting elements.

13. The environment controlled chamber of claim 1 wherein the control system is configured to control the plurality of infrared lighting elements to selectively apply the heating to assist with preventing condensation on petri dishes stored within the insulated cabinet based on maximum intensity of lighting within the environment controlled chamber.

14. A method for controlling an environment controlled chamber to assist with preventing condensation on an inside of a top cover of a petri dish stored within the environment controlled chamber, the method comprising steps of:
receiving through a user interface parameters for controlling the environment controlled chamber where the parameters including a temperature setting, and a light control setting associated with activating and deactivating lighting elements within the environment controlled chamber; and
activating infrared lighting elements within the environment controlled chamber based on the temperature setting and the light control setting and while cooling the environment controlled chamber to increase temperature differential between a surface temperature of the top cover of the petri dish and an ambient dew point inside the petri dish.

15. The method of claim 14 wherein the infrared lighting elements are infrared LEDs.

16. The method of claim 14 further comprising acquiring sensor measurements from a plurality of sensors and wherein the activating the infrared lighting elements is further based on the sensor measurements.

17. An environment controlled chamber system, comprising:
an insulated cabinet;
a refrigeration system adapted for cooling the insulated cabinet;
a control system;
a plurality of light emitting diodes (LEDs) for providing light within a compartment of the insulated cabinet; and
a plurality of infrared (IR) light emitting diodes (LEDs) for providing heating within the insulated cabinet;
wherein the control system is configured to control the refrigeration system and the LEDs to provide environmental conditions according to user settings for promoting photosynthetic tissue culture growth;
wherein the control system is configured to control the plurality of IR LEDs to apply heating to assist with preventing condensation on petri dishes stored within the insulated cabinet by increasing differences between surface temperatures of the petri dishes and dew points inside the petri dishes.

18. The environment controlled chamber system of claim 17 wherein the control system is configured to apply infrared levels based on maximum intensity of lighting in the compartment of the insulated cabinet.

19. The environment controlled chamber system of claim 17 wherein the control system is configured to control the LEDs to provide the environmental conditions simulating a day to night transition and wherein the control system activates the IR LEDs based on timing of the day to night transition.

20. The environment controlled chamber system of claim 17 wherein the control system is configured to activate the IR LEDs in order to add heat within the insulated cabinet while cooling the insulated cabinet to thereby limit rate of temperature change within the insulated cabinet to assist with preventing the condensation on the petri dishes stored within the insulated cabinet.

21. The environment controlled chamber system of claim 17 further comprising a user interface configured to receive input from a user to establish the user settings.

22. The environment controlled chamber system of claim 17 wherein a material is integrated into a medium of the petri dishes to increase heating effects of the plurality of IR LEDs.

23. The environment controlled chamber of claim 17 wherein a material is integrated into the petri dishes to increase heating effects of the plurality of IR LEDs.

24. The environment controlled chamber of claim 17 wherein a material is integrated into the compartment to increase heating effects of the plurality of IR LEDs.

25. An environment controlled chamber, comprising:
an insulated cabinet;
a refrigeration system adapted for cooling the insulated cabinet;
a control system;
a plurality of lighting elements within a compartment of the insulated cabinet; and
a plurality of infrared elements for providing heating within the insulated cabinet;
wherein the control system is configured to control the refrigeration system and the lighting elements to provide environmental conditions according to user settings;
wherein the control system is configured to control the plurality of infrared elements to selectively apply heating to assist with preventing condensation on petri dishes stored within the insulated cabinet.
